(19) United States
(12) Patent Application Publication
(10) Pub. No.: US 2019/0142309 A1
(43) Pub. Date: May 16, 2019

(54) HEXAGONAL NANOFLUIDIC MICROCHANNELS FOR BIOFLUID SENSING DEVICES

(71) Applicants: Eccrine Systems, Inc., Cincinnati, OH (US); University of Cincinnati, Cincinnati, OH (US)

(72) Inventors: Jason Heikenfeld, Cincinnati, OH (US); Ryan Michael Norton, Lexington, KY (US); Nicholas Twine, Cincinnati, OH (US); Michael Charles Brothers, Lebanon, OH (US)

(73) Assignees: Eccrine Systems, Inc., Cincinnati, OH (US); University of Cincinnati, Cincinnati, OH (US)

(21) Appl. No.: 16/206,095

(22) Filed: Nov. 30, 2018

Related U.S. Application Data
(63) Continuation-in-part of application No. 15/958,725, filed on Apr. 20, 2018, now abandoned.
Application No. 15/958,725 is a continuation-in-part of application No. 15/746,452, filed on Jan. 22, 2018.
(60) Provisional application No. 62/633,210, filed on Feb. 21, 2018.
(60) Provisional application No. 62/196,541, filed on Jul. 24, 2015.
(60) Provisional application No. 62/208,171, filed on Aug. 21, 2015.
(60) Provisional application No. 62/592,685, filed on Nov. 30, 2017.

(51) Int. Cl. A61B 5/1477; A61B 5/00; A61B 5/145

(52) U.S. Cl. CPC A61B 5/1477; A61B 5/1451; A61B 5/6833

(57) ABSTRACT

The disclosed invention provides a biofluid collection device configured with an open microfluidic network, which facilitates nanoliter-scale biofluid collection and transport for biosensing applications. In one embodiment, a biofluid sensing device placed on the skin for measuring a characteristic of an analyte in sweat includes one or more biofluid sensors and a hexagonal open microfluidic network biofluid collector. The disclosed collector provides a volume-reduced pathway for sweat biofluid between the one or more sensors and sweat glands when the device is positioned on the skin. In another embodiment, a biofluid collector includes a network of microchannels comprising three or more repeatedly intersecting channels that provide redundant pathways for biofluid transport. Embodiments of the disclosed invention are also directed to highly stable peptide-based self-assembled monolayers (SAM) and methods of making the SAMs. In some embodiments, the peptide-based SAM is formed on a component of a biofluid sensing device.

### CROSS-REFERENCE TO RELATED APPLICATIONS

This application is a continuation-in-part of U.S. application Ser. No. 15/958,725, filed Apr. 20, 2018, and claims priority to U.S. Provisional Application No. 62/592,685, filed Nov. 30, 2017, U.S. application Ser. No. 15/746,452, filed Jan. 22, 2018; U.S. Provisional Application No. 62/633,210, filed Feb. 21, 2018; as well as PCT/US16/43771, filed Jul. 23, 2016, the disclosures of which are hereby incorporated by reference herein in their entirety.

### STATEMENT REGARDING FEDERALLY SPONSORED RESEARCH OR DEVELOPMENT

This invention was made with government support under NSF ECCS-1608275 awarded by the National Science Foundation. The government has certain rights in the invention.

### BACKGROUND OF THE INVENTION

This application has specification that builds upon Twine, N., et al., “Open Nanofluidic Films with Rapid Transport and No Analyte Loss for Ultra-Low Sample Volumes,” Lab on a Chip, 2018, which is hereby incorporated by reference herein in its entirety.

Sweat contains many of the same biomarkers, chemicals, or solutes that are carried in blood and can provide significant information enabling one to diagnose illness, health status, exposure to toxins, performance, and other physiological attributes even in advance of any physical sign. Furthermore, sweat itself, the action of sweating, and other parameters, attributes, solutes, or features on, near, or beneath the skin can be measured to further reveal physiological information. Of the other physiological fluids (biofluids) used for biological monitoring (e.g., blood, urine, saliva, tears, interstitial fluid, etc.), sweat has arguably the least predictable sampling rate in the absence of technology. However, with proper application of technology, sweat can be made to outperform other non-invasive or less invasive biofluids in predictable sampling.

However, the state of art in sweat bio monitoring is in need of additional devices and methods to properly reduce the dead volume between sensors and skin. Reducing dead volume reduces the amount of biofluid required to reliably transport a biofluid sample across sensors, and reduces the opportunity for newer sweat to mix with older sweat, which mixing confounds chronological measurements. Further, transporting a very low volume of biofluid to sensors is critical to achieve fast sampling times, or for sampling during intervals with very low sweat rates. In addition, it also may be critical for prolonged stimulation (i.e., in order to minimize stimulation), and for improving biomarker measurements where a low sweat rate is required to ensure correlation between biomarker concentrations in sweat and those in blood.

While techniques for transporting microliter sample volumes to sensors for analyte sensing is now technologically mature, current solutions in the art are often ill-suited to applications in the nanoliter regime (<100 nL). Challenges associated with nanoliter transport to sensors as well as interface with sensors include difficulties in sensor integration with the transport means, increased resistance to fluid flow, and prohibitive amounts of analyte exchange between the sample and the transport medium. For example, in sweat sensing applications recent work to reduce sample volumes by using an ˜8 μL microchannel and a sweat collection area of ˜0.1 cm² still requires an 8.5-hour collection time at conventional sweat generation rates (˜1 nL/min/gland). Similarly, existing wicking materials have shown inadequacy for sweat sensing applications due to excessive analyte exchange. For example, Rayon™ has advantageous properties for reducing sample volume, since its structure allows fluid transport along wicking nano-grooves, without the need to wet the entire material. However, analyte exchange with Rayon fabric is so prevalent that even high concentration analytes such as electrolytes (10's mMol), can become sufficiently depleted in the sweat sample to prevent rapid sensing of concentration changes. Other widely used wicking materials are even more problematic for low concentration analytes, e.g., PDMS readily adsorbs hydrophobic small molecules, such as hormones, that are found in nM unbound concentrations in sweat.

Other microfluidic structures disclosed in the art fall short of the capabilities of the disclosed invention. For example, U.S. Pat. No. 7,682,817 B2, from Cohen, D., et al. (“Cohen”) discloses a microfluidic wicking device for clearing a fluid sample from a test area. Cohen's device includes a fluid collector that delivers a fluid sample to a testing area, and then a plurality of microfluidic channels carries the fluid sample away from the test area at a controlled rate. Cohen therefore solves a fundamentally different problem than is solved by the disclosed invention, which efficiently delivers low volume samples from skin to an analyte sensor. Structurally, Cohen is also dissimilar from the disclosed invention. Cohen's microchannels are arranged in a radial fashion to disperse fluid collected in a small area to a large area. By contrast, the disclosed invention features multiple intersecting paths that move fluid from a large area to a small area (an analyte sensor). The analogous portion of Cohen, e.g., the input channel 12 from FIG. 1 therein, is described as having embodiments that include a network of T-junctions or Y-junctions, however no hexagonal network of sample collection channels is disclosed, nor is any particular function for these alternate embodiments described. And while Cohen discloses a microchannel with a hexagonal cross-section it does not discuss a network of channels whose layout forms a series of hexagonal structures. U.S. patent application Ser. No. 14/384,764 from Azioune, et aL, (“Azioune”) discloses improving the contact angle of microfluidic channels through physical treatments, namely irradiation of the polymer substrate, but Azioune does not mention peptide functionalization coatings, and in fact teaches way from such chemical treatments to improve contact angle, see Para. 0005. Other art in the field discloses various microfluidic pumps, but these devices are expensive, complex, and unsuitable for wearable biofluid sensing. See, e.g., U.S. patent application Ser. No. 10/886,408 from Blackburn, G., (disclosing vacuum, pressurized gas, electroosmotic, electrohydrodynamic and electrokinetic fluid pumps); and U.S. patent application Ser. No. 11/776,351, from Santini J., et al. (disclosing powered pumps with mechanical moving parts). Neither of these references discuss a wicking pump of the disclosed invention, which is desirable to reduce device size, expense, and complexity.

Therefore, what is needed are materials and methods to provide biofluid transport and sensor interface at the nanoliter scale that allow for responsive and continuous sensing of low concentration analytes. Further, new, low-cost SAMs composed of monomers that are safe for contact with human skin, and that maintain a low contact angle over the course of days are also required.

### SUMMARY OF THE INVENTION

The disclosed invention provides a biofluid collection device configured with an open microfluidic network, which facilitates nanoliter-scale biofluid collection and transport for biosensing applications. A hexagonal network is taught, but other networks (square, triangular, random) are possible within the disclosed invention. In one embodiment, a biofluid sensing device placed on the skin for measuring a characteristic of an analyte in sweat includes one or more biofluid sensors and a hexagonal open microfluidic network biofluid collector. The disclosed collector provides a volume-reduced pathway for sweat biofluid between the one or more sensors and sweat glands when the device is positioned on the skin. In another embodiment, a biofluid collector includes a network of microchannels comprising three or more repeatedly intersecting channels that provide redundant pathways for biofluid transport. Embodiments of the disclosed invention are also directed to highly stable peptide-based self-assembled monolayers (SAM) and methods of making the SAMs. In some embodiments, the peptide-based SAM is formed on a component of a biofluid sensing device.

### BRIEF DESCRIPTION OF THE DRAWINGS

The disclosed invention will be further appreciated in light of the following descriptions and drawings in which:

FIG. 1 depicts at least a portion of a device comprising the disclosed invention.

FIG. 2 depicts at least a portion of a device comprising an open nanofluidic film for low volume biofluid transport.

FIG. 3 depicts at least a portion of a device comprising an open nanofluidic film for low volume biofluid transport.

FIG. 4 depicts at least a portion of a device comprising an open nanofluidic film for low volume biofluid transport.

FIG. 5 depicts at least a portion of a device comprising an open nanofluidic film for low volume biofluid transport.

FIG. 6 depicts at least a portion of a microchannel cross section of the disclosed invention.

FIGS. 7 and 7A-7E are top cross-sectional views of a channel junction in the hexagonal wick of FIG. 4 as fluid moves through the junction.

FIGS. 7F-7I are side cross-sectional views of the channel junction of FIGS. 7A, 7B, 7C, and 7E, respectively.

FIG. 8 is a chart showing the contact angle degradation over time for three different peptide-based SAMs stored in three different conditions.

### DEFINITIONS

“Continuous monitoring” means the capability of a device to provide at least one measurement of biofluid determined by a continuous or multiple collection and sensing of that measurement or to provide a plurality of measurements of biofluid over time.

As used herein, “interstitial fluid” or “tissue fluid” is a solution that bathes and surrounds tissue cells. The interstitial fluid is found in the interstices between cells. Embodiments of the disclosed invention measure analytes from interstitial fluid found in the skin and, particularly, interstitial fluid found in the dermis. In some cases where interstitial fluid is emerging from sweat ducts, the interstitial fluid contains some sweat as well, or alternately, sweat may contain some interstitial fluid.

As used herein, “biofluid” may mean any human biofluid, including, without limitation, sweat, interstitial fluid, blood, plasma, serum, tears, and saliva. For sweat sensing applications as generally discussed herein, biofluid has a narrower meaning, namely, a fluid that is comprised mainly of interstitial fluid or sweat as it emerges from the skin.

“Chronological assurance” means a sampling rate or sampling interval for measurement(s) of biofluid, or solutes in biofluid, at which measurements can be made of new biofluid or its new solutes as they originate from the body. Chronological assurance may also include a determination of the effect of sensor function, or potential contamination with previously generated biofluid, previously generated solutes, other fluid, or other measurement contamination sources for the measurement(s).

As used herein, “biofluid sampling rate” or “sampling rate” is the effective rate at which new biofluid, originating from pre-existing pathways, reaches a sensor that measures a property of the fluid or its solutes. Sampling rate is the rate at which new biofluid is refreshed at the one or more sensors and therefore old biofluid is removed as new fluid arrives. In one embodiment, this can be estimated based on volume, flow-rate, and time calculations, although it is recognized that some biofluid or solute mixing can occur. Sampling rate directly determines or is a contributing factor in determining the chronological assurance. Times and rates are inversely proportional (rates having at least partial units of 1/seconds), therefore a short or small time required to refill sample volume can also be said to have a fast or high sampling rate. The inverse of sampling rate (1/s) could also be interpreted as a “sampling interval(s)”. Sampling rates or intervals are not necessarily regular, discrete, periodic, discontinuous, or subject to other limitations. Like chronological assurance, sampling rate may also include a determination of the effect of potential contamination with previously generated biofluid, previously generated solutes (analytes), other fluid, or other measurement contamination sources for the measurement(s). Sampling rate can also be in part determined from solute generation, transport, advective transport of fluid, diffusion transport of solutes, or other factors that will impact the rate at which new sample will reach a sensor and/or is altered by older sample or solutes or other contamination sources.

As used herein, “sample generation rate” is the rate at which biofluid is generated by flow through pre-existing pathways. Sample generation rate is typically measured by the flow rate from each pre-existing pathway in nL/min/pathway. In some cases, to obtain total sample flow rate, the sample generation rate is multiplied by the number of pathways from which the sample is being sampled. Similarly, as used herein, “analyte generation rate” is the rate at which solutes move from the body or other sources toward the sensors.

“Analyte” means a substance, molecule, ion, or other material that is measured by a biofluid sensing device.

“Molecule” means a group of two or more atoms joined by chemical bonds, and is not limited to such compounds with a neutral electrical charge.

“Measured” may mean an exact or precise quantitative measurement and can include broader meanings such as, for example, measuring a relative amount of change of something. Measured can also mean a binary measurement, such as ‘yes’ or ‘no’ type measurements.

“Biofluid sensor” means any type of sensor that measures a state, presence, flow rate, solute concentration, solute presence, in absolute, relative, trending, or other ways in a biofluid. Biofluid sensors can include, for example, potentiometric, amperometric, impedance, optical, mechanical, antibody, peptide, aptamer, or other means known by those skilled in the art of sensing or biosensing.

“EAB sensor” means an electrochemical aptamer-based biosensor that is configured with multiple aptamer sensing elements that, in the presence of a target analyte in a biofluid sample, produce a signal indicating analyte capture, and which signal can be added to the signals of other such sensing elements, so that a signal threshold may be reached that indicates the presence of the target analyte.

As used herein, “sample volume” is the fluidic volume in a space that can be defined multiple ways. Sample volume may be the volume that exists between a sensor and the point of generation of biofluid sample. Sample volume can include the volume that can be occupied by sample fluid between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

“Volume-reducing component” means any component, material, element, or feature of the present disclosure that facilitates the creation of a volume-reduced pathway.

“Volume-reduced pathway” means a sample volume that has been reduced by the addition of a material, device, layer, or other component, which therefore decreases the sampling interval for a given sample generation rate. Specific to the instant disclosure, a volume reduced pathway refers to any combination of elements disclosed herein that at least in part uses wicking pressure to enable the formation of the volume reduced pathway. For example, a volume reduced pathway could be created in the space between a biofluid collector and skin by wicking biofluid through this space. The disclosed invention may benefit from additional methods to reduce the sample volume, but if the term volume-reduced pathway is used herein, then wicking pressure must, at least in part, enable or create the volume-reduced pathway.

“Microfluidic components” means channels in polymer, textiles, paper, or other components known in the art of microfluidics for guiding movement of a fluid or at least partial containment of a fluid.

“Nanofluidic wicking” means channels that transport biofluids on a nanoliter L) scale.

“Peptide” means short chains of amino acid monomers, i.e., less than around 50 amino acid monomers, linked by amide bonds.

### DETAILED DESCRIPTION OF THE INVENTION

The disclosed invention includes a design for a hexagonal wick (“hex wick”) which addresses major challenges in nanoscale biofluid transport and sensing through the incorporation of several innovative features: (1) the wick achieves an effective wicking film thickness of ˜1 μm (<100 nL/cm²) through a hexagonal network of ˜10×15 μm open channels that comprise ˜10% of the open surface area; (2) analyte exchange with the wick is substantially prevented by use of a thin analyte exchange blocking coating (e.g., gold, silver, mercury, iron, graphene); (3) rapid wicking transport through rectangular microchannels reduces resistance to fluid flow as compared to traditional wicking materials; (4) ease of manufacture; (5) hydrophilicity provided through a shelf-stable and biologically safe peptide surface modification; (6) hydrophilicity allows omnidirectional wicking beyond corner junctions as compared to traditional linear wicking; (7) specific to sweat biosensing, the wick also reduces the dead volume against the skin surface which reduces contamination from the stratum corneum.

To clarify the proper numerical values or representations of sampling rate for sweat and therefore chronological assurance, sweat generation rate and sweat volumes will be described in detail. From Dermatology: an illustrated color text, 5th ed., the maximum sweat generated per person per day is 10 L, which on average is 4 μL per gland maximum per day, or about 3 nL/min/gland. This is about 20× higher than the minimum sweat generation rate. The maximum stimulated sweat generation rate according to Buono 1992, J. Derm. Sci. 4, 33-37, “Cholinergic sensitivity of the eccrine sweat gland in trained and untrained men,” the maximum sweat generation rate by pilocarpine stimulation is about 4 nL/min/gland for untrained men and 8 nL/min/gland for trained (exercising often) men. Sweat stimulation data from “Pharmacologic responsiveness of isolated single eccrine sweat glands,” by K. Sato and F. Sato, Am. Physiological Society, Jul. 30, 1980, suggests a sweat generation rate up to about 5 nL/min/gland is possible with stimulation, and several types of sweat stimulating substances are disclosed (the data was for extracted and isolated monkey sweat glands, which are very similar to human ones). For simplicity, we can assume for calculations in the present disclosure (without so limiting the disclosure), that the minimum sweat generation rate is about 0.1 nL/min/gland, and the maximum sweat generation rate is about 5 nL/min/gland, which is about a 50× difference between the maximum and minimum rates.

Based on the assumption of a sweat gland density of 100/cm², a sensor that is 0.55 cm in radius (1.1 cm in diameter) would cover about 1 cm² area, or approximately 100 sweat glands. Next, assume a sweat volume under a skin-facing sensor (space between the sensor and the skin) of 100 μm average height or 100E-4 cm, and that same 1 cm² area, which provides a sweat volume of 100E-4 cm³ or about 100E-4 mL or 10 μL of volume. With the maximum sweat generation rate of 5 nL/min/gland and 100 glands, it would require 20 minutes to fully refresh the sweat volume (using first principles/simplest calculation only). With the minimum sweat generation rate of 0.1 nL/min/gland and 100 glands, it would require 1000 minutes or ˜17 hours to refresh the sweat volume. Because the flow is not entirely centered, according to Sonner, et al., in Biomicreuidics, May 15, 2015; 9(3):031301. doi: 10.1063/1.4921039, the time to fully refresh the sweat volume (i.e., new sweat replaces all old sweat) could be six times longer or more. For slow sweat flow rates, back-diffusion of analytes and other confounding factors could make the effective sampling interval even larger. Clearly, conventional wearable sweat sensing approaches with large sweat volumes and slow sampling rates would find continuous sweat sample monitoring to be a significant challenge.

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

Sweat stimulation, or sweat activation, can be achieved by known methods. For example, sweat stimulation can be achieved by simple thermal stimulation, chemical heating pad, infrared light, by orally administering a drug, by intradermal injection of drugs such as carbachol, methylcholine or pilocarpine, and by dermal introduction of such drugs using iontophoresis, by sudo-motor-axon reflex sweating, or by other means. A device for iontophoresis may, for example, provide direct current and use large lead electrodes lined with porous material, where the positive pole is dampened with 2% pilocarpine hydrochloride or carbachol and the negative one with 0.9% NaCl solution. Sweat can also be controlled or created by asking the device wearer to conduct or increase activities or conditions that cause them to sweat.

The present disclosure applies at least to any type of biofluid sensing device that stimulates sweat, measures biofluid, sample generation rate, chronological assurance, its solutes, solutes that transfer into biofluid from skin, a property of or things on the surface of skin, or properties or things beneath the skin. The disclosed invention may include at least one sensor that is specific to an analyte in biofluid. To clarify further, just measuring biofluid conductivity is not specific to one analyte because it measures the sum of conductance contributed by all ionic solutes in the biofluid. However, an ion-selective electrode configured to detect potassium is a sensor specific to one analyte. As an additional example, a sensor for biofluid cortisol that only has interference (non-specificity) to estrogen, would still be specific to one analyte as described herein, since there are many device applications in which estrogen concentrations are static, but cortisol concentrations would change, making the sensor effectively specific to cortisol. Any suitable sensor may be used in the disclosed invention (e.g., ion-selective, enzymatic, antibody, aptamer, optical, electrical, mechanical, etc.). The disclosure applies to biofluid sensing devices with various configurations including patches, bands, straps, portions of clothing, wearables, or any suitable mechanism that reliably brings sweat stimulating, biofluid collecting, and/or biofluid sensing technology into intimate proximity with biofluid as it is generated. Some embodiments use adhesives to hold the device near the skin, but devices may also be secured by another suitable mechanism, such as a strap or helmet suspension.

Certain embodiments of the disclosure describe sensors as simple individual elements. It is understood that many sensors require two or more electrodes, reference electrodes, or additional supporting technology or features that are not captured in the description herein. Sensors are preferably electrical in nature, but may also include optical, chemical, mechanical, or other known biosensing mechanisms. Sensors can be in duplicate, triplicate, or more, to provide improved data and readings. Sensors may be referred to by what the sensor is sensing, for example: a biofluid sensor; an impedance sensor; a biofluid volume sensor; a biofluid generation rate sensor; or a solute generation rate sensor. Certain embodiments of the disclosed invention show sub-components that may require additional obvious sub-components for use of the device in various applications (such as a battery), and for purpose of brevity and focus on inventive aspects are not explicitly shown in the diagrams or described in the embodiments of the present disclosure. As a further example, many embodiments of the disclosed invention may benefit from mechanical or other means to keep the devices or sub-components firmly affixed to skin or to provide pressure facilitating constant contact with skin or conformal contact with ridges or grooves in skin, as are known to those skilled in the art of wearable devices, patches, bandages, or other technologies or materials that are affixed to skin. Such means are included within the spirit of the disclosed invention. The present application has specification that builds upon PCT/US13/35092, the disclosure of which is hereby incorporated herein by reference in its entirety.

Embodiments of the present invention also include highly stable peptide-based self-assembled monolayers (SAM) as functionalization coatings that improve fluid contact angles within the disclosed device. Such peptide functionalization coatings enable efficient wicking transport of biofluid. As used herein, peptides are polymers of two or more amino acids. The number of amino acids in a peptide can range from two to tens, to hundreds, to thousands. Peptides may have a secondary structure in the shape of an alpha helix, a beta sheet, or a random coil. Shorter peptides (e.g., less than 20 amino acids) can have predictable secondary structure based on the primary structure and thus may be utilized to generate a predictable structure in high yield. One of the amino acids that can be incorporated is cysteine, which contains a thiol. Thiols (also referred to as ‘mercaptans’) are a sulfhydryl group that is attached to various molecular structures such as an alkyl or other organic substitute, allowing it to form a SAM on a structure.

Most SAMs contain only one thiol. Many thiols have been created that have a hydroxyl group, a carboxylate, or another highly hydrophilic moiety at the interface with water, such as 3-mercapto-propanesulfonate (MPS), thioglycolic acid (TGA), and others, but most thiols have one connection to the substrate holding the entire molecule down, making them susceptible to removal by transient gold oxidation. As such, they degrade over the course of hours to days. Dithiols and trithiols that are commercially available can be used to generate SAMs with increased stability, as two or three thiols must be removed simultaneously to remove the molecule from the gold surface. However, these dithiols or trithiols that are commercially available are often limited in scope, cost prohibitive, are costly to modify, and more importantly, with the exception of lipoic acid, are not composed of FDA-approved monomers.

Peptide-based SAMs can have two or more engineered thiols that bind covalently to the substrate, and/or engineered amines that bind non-covalently to the substrate surface and can incorporate both native amino acids (i.e., standard amino acids, encoded by the naturally occurring universal genetic code) and non-native amino acids (i.e., non-standard amino acids that may include N-hydroxy-succinimide groups, malemide groups, alkyne groups, and/or azide groups), enabling the changing of the hydrophilicity or the hydrophobicity of the water-peptide interface. In an aspect of the present invention, different chemistries can be incorporated into the peptide-based SAM layer on a sensor that can directly link to a peptide or deoxyribonucleic acid (DNA) sequence, or protein of interest. The peptide chemistry and the influence of primary structure on secondary structure can influence the design of these peptide-based SAMs. Peptide-based SAMs provide very stable monolayers on substrates or blocking coatings made of, for example, gold, silver, mercury, iron, graphene, etc.

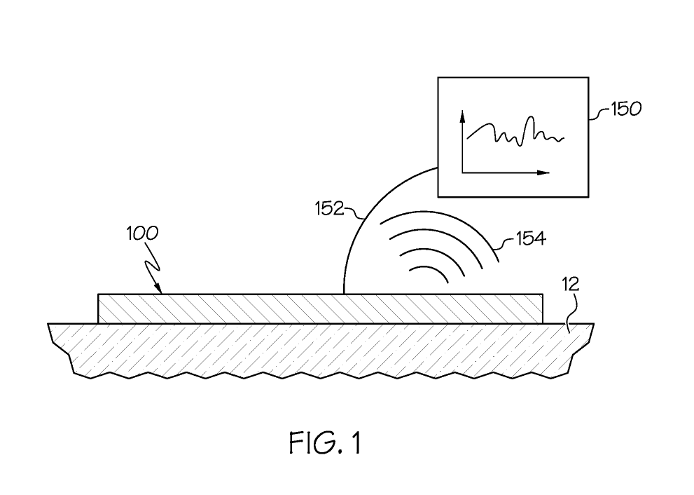

With reference to FIG. 1, a biofluid sensing device 100 is placed on or near skin 12. In an alternate embodiment, the biofluid sensing device may be simply fluidically connected to skin or regions near skin through microfluidics or other suitable techniques. The device 100 is in wired communication 152 or wireless communication 154 with a reader device 150. In some embodiments, reader device 150 may be a smart phone or portable electronic device. In alternate embodiments, device 100 and reader device 150 can be combined. In further alternate embodiments, communication 152 or 154 is not constant and could be a one-time data transmission from device 100 once it has completed its measurements of biofluid.

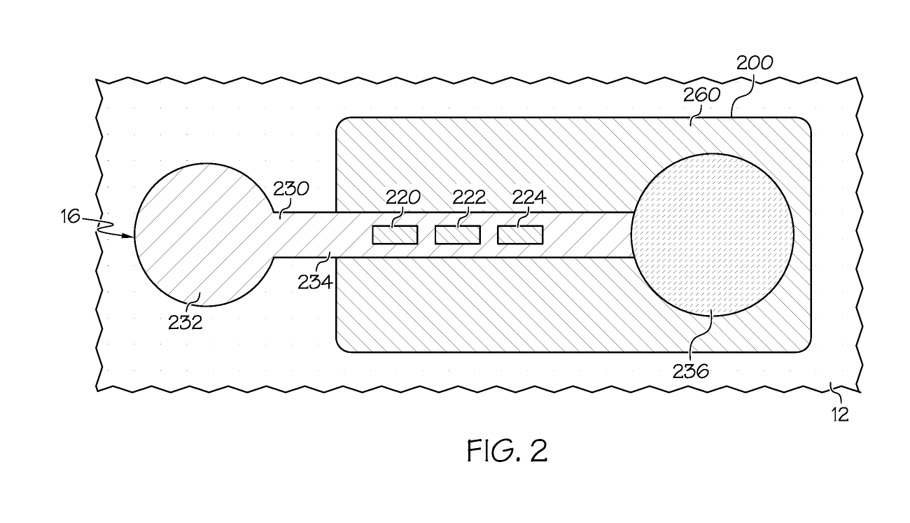

FIG. 2 depicts an overhead view of a wearable biofluid sensing device 200 as it is worn on skin 12. The device includes a fluid impermeable substrate 260, made from, e.g., PET, PVC; and a microfluidic wick 230, which includes a wicking collector 232 and a wicking coupler 234. The wicking coupler 234 may be constructed of a polymer, paper, textile, rayon, or other suitable material for transporting the biofluid sample across one or more biofluid sensors 220, 222, 224 and facilitating the interface between the biofluid sample and sensors. The microfluidic wick 230 is in fluidic communication with the skin 12, a wicking pump 236, and the one or more sensors 220, 222, 224.

The wicking pump 236 is constructed of paper, or may be an absorbent hydrogel, a desiccant, or other material suitable for drawing a biofluid sample across and away from the sensors. The wicking pump 236 should have sufficient volume to sustain operation of the device throughout the application's intended duration (i.e., it should not become saturated during device operation). For example, if the device is to be used for 24 hours, then neither microfluidic wick 230 nor the wicking pump 236 should become fully saturated with sweat during the 24 hours of operation. In some embodiments, microfluidic wick 230 and wicking pump 236 may be the same material or component.

The sensors include one or more analyte specific sensors 220, 222, e.g., ion-selective electrode sensors, electrochemical aptamer-based sensors, amperometric, or enzymatic sensors. Some embodiments also include one or more secondary sensors 224, which may be, e.g., volumetric sweat rate, micro-thermal flow rate, GSR, biofluid conductivity, impedance or capacitance sensors for skin contact measurement, or a temperature sensor.

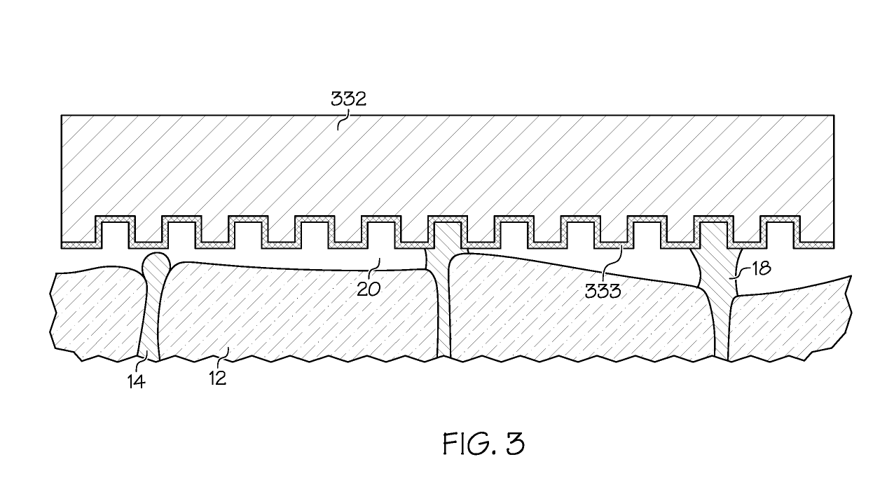

With reference to FIG. 3, which depicts the wicking collector 232 of FIG. 2 as viewed from the direction of the arrow 16, the wicking collector 332 interacts with the skin 12 of the device wearer. The wicking collector 332 is comprised of a polymer having a skin-facing surface that contains a plurality of interconnected microchannels 333 arranged in a hexagonal pattern. The microchannels have dimensions of 10 μm width and 15 μm height (width-to-height ratio of 1:1.5), but may have other dimensions, e.g., a width and/or height of 5 μm, 10 μm, 15 μm, 20 μm, 25 μm, or 30 μm, or different width-to-height ratios, e.g., ≥1:3, ≥1:2, ≥1:1, ≥1:1.5, ≥1:2, ≥1:3, ≥1.5:1, ≥2:1, or ≥3:1. The microchannels preferably have substantially square (not rounded) corners. The channels may be manufactured in a variety of ways, such as laser etching the channels into the polymer. Other techniques include casting the channels by pouring the polymer into a mold bearing the desired pattern, and then curing the polymer. The wicking collector 332 may be constructed of any material that allows adhesion of the analyte exchange blocking coating, and can achieve the required geometric shape. Alternatively, the wicking collector 332 may be constructed of a simple hydrophilic polymer, or a polymer, e.g., PET, that is treated or coated to be hydrophilic or super-hydrophilic, such as by coating with a nano-silica, or a hydrogel such as agar. Between the skin 12 and the wicking collector 332, is a wicking space or dead volume 20. As sweat 14 leaves the skin, it first forms droplets, and when sufficient sweat is produced by the sweat gland, it wets 18 the wicking collector 332, and enters the microchannels 333, where it is transported to the wicking coupler (not shown).

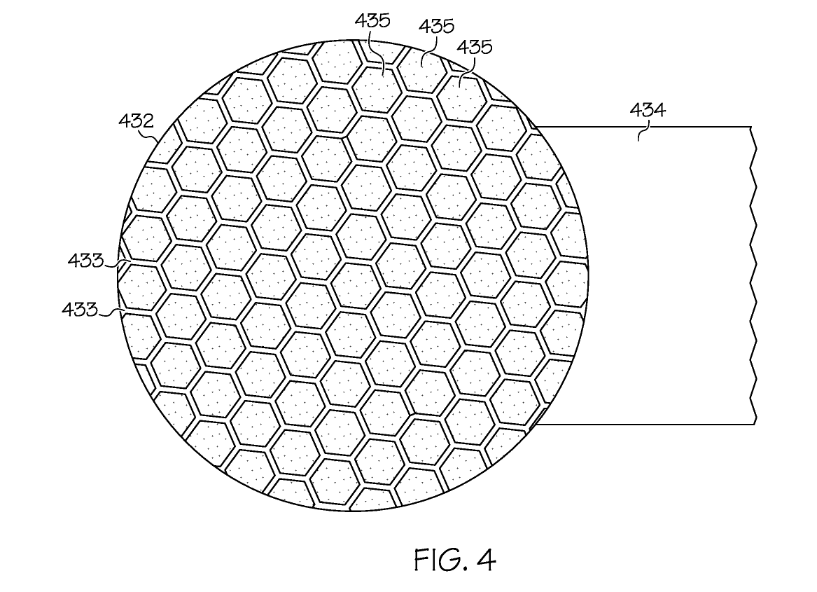

With reference to FIG. 4, the underside of wicking collector 232 of FIG. 2 is depicted. The skin-facing side of the wicking collector 432, comprises a plurality of open interconnecting microchannels 433 that create a plurality of hexagonal structures 435 between the channels. The hexagonal structures 435 and microchannels 433 have a hydrophilic analyte exchange blocking coating, e.g., a sputter-deposited 10 nm gold coating, to reduce contamination from skin. The microchannels create a hexagonal network of open surface channels and intervening hexagonal structures, a hex wick, which satisfies a number of requirements for nanoscale biofluid transport, that include: transport of ultra-low biofluid volumes; minimized surface-area to volume; no or negligible analyte exchange with the hex wick, and simplicity of manufacture. Regarding simplicity of manufacture, large sheets of hex wicks can be fabricated, and then cut to size and laminated against other components, such as the wicking coupler 434, sensors (not shown), or additional hex wicks (not shown) to construct a biofluid sensing device.

The hex wick as disclosed also provides a number of advantages over other biofluid collection configurations. For example, compared to a sweat collector with a single continuous channel, the hex wick provides multiple redundant paths for a biofluid sample to reach the sensors. If the single channel were to suffer a blockage, break, or other defect, the wicking and biofluid transport capability of the entire wicking collector could be disrupted. A hex wick, however, provides redundancy in potential wicking paths, meaning that a broken sub-channel will not prevent the network from wicking and transporting biofluid. Therefore, embodiments of the disclosed invention may include a network of at least partially redundant wicking pathways.

Another advantage of the disclosed hex wick is the ability to provide greater contact area between wicking channels and sweat gland openings relative to existing biofluid collector materials. For example, a simple textile biofluid collector with random fiber arrangement (e.g., non-woven) could have areas with poor local contact to skin, and therefore in some areas would require more biofluid volume in order to allow wicking connection between the opening of a sweat gland on the skin surface and the textile. The disclosed hex wick, however, can be precisely configured so that there is no more than 500 μm, and preferably no more than 100 μm, distance between adjacent wicking pathways in the hex wick, thereby providing consistently small distances between wicking pathways and sweat glands, and in turn an overall reduction in biofluid volume required by the device.

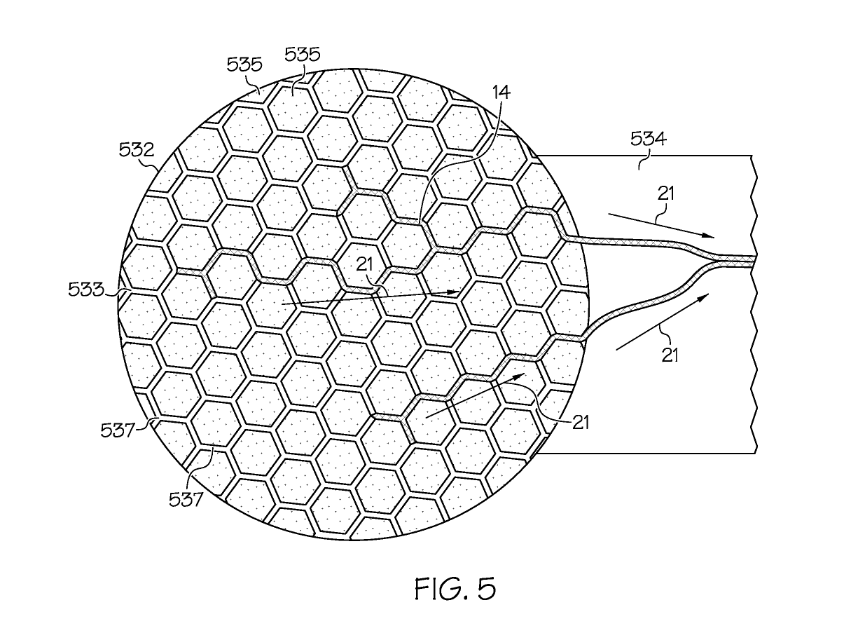

With reference to FIG. 5, the underside of the wicking collector 432 of FIG. 4 is depicted under active sweating conditions. As sweat 14 wets into the microchannels 533, it wicks along the channel pathways in the direction of the arrows 21 to the wicking coupler 534, and to the sensors (not shown). For simplicity of illustration, example preferred pathways 21 are shown, not shown is wicking of the fluid in other directions as well (all or most of the microchannels 533 can potentially become partially or fully wetted during operation). The mechanics of fluid transport in the hex wick are quite sophisticated, particularly due to the divergent capillary dimensions of the microchannels 533 that exist at the connecting junctions 537. Several wicking principles are required to characterize the fluid flow through the hex wick, and will be described here in the order of difficulty for achieving continuous wicking through the microchannels. The easiest model available is capillary flow through microchannels, wherein the channels are modeled as open u-channels with perfectly square corners. However, due to manufacturing difficulty, the microchannels will have somewhat rounded corners. As a result, more complex models will have to be used, including modeling capillary flow through open u-channels with rounded corners, and modeling the flow of capillary filaments propagating along open u-channels with rounded corners.

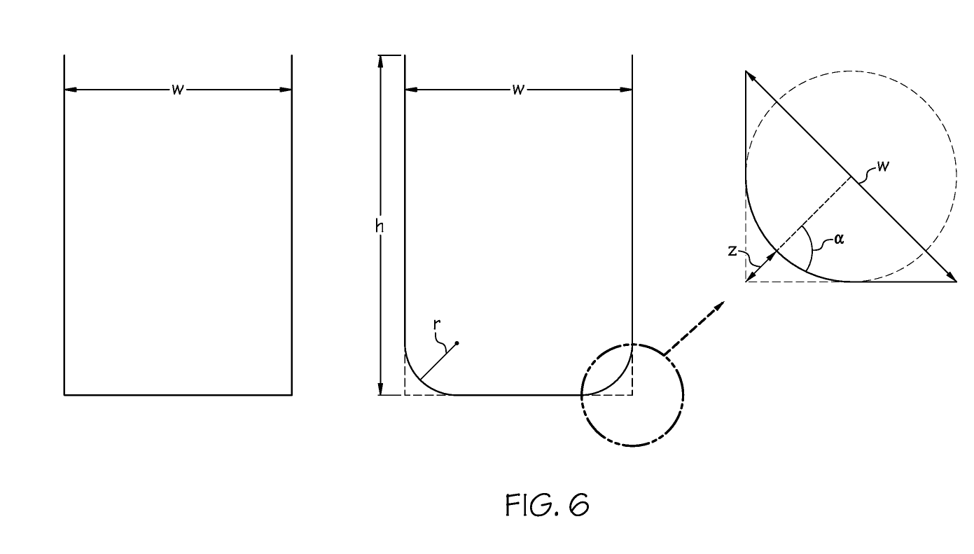

With reference to FIG. 6, the simplest model of capillary flow through a hex wick microchannel is to treat the open u-channel as a combination of two perfectly square corners. The u-channel corner wicking is determined by the channel aspect ratio: width (w) and height (h), and Young's contact angle (θ). For an open u-channel with perfectly square corners, the condition for capillary now is:

$$\frac{w}{2 h + w} < cos θ .$$

Thus, for an aspect ratio of 1.5 (10 μm width and 15 μm height), the contact angle necessary to satisfy capillary flow is <75°. Maintaining such a low contact angle is trivial, but real-world fabrication methods will likely have corner rounding with a radius (r), resulting in a more challenging condition for capillary flow:

$$\frac{w}{2 h + w} + \frac{wr ( 4 - π )}{2 h + {w}^{2}} < cos θ .$$

Using this equation, even where corner rounding is worst-case, i.e., the corner radius is equal to the 10 μm width of the channel, the contact angle necessary for capillary flow is 66°, which is also trivial to achieve with many hydrophilic materials.

However, because the hex wick has divergent capillary geometries at the channel junctions, a third more difficult requirement exists: unless more difficult-to-make high-aspect-ratio channels are utilized, capillary filaments along the corners are necessary to promote continuous wicking. The requirement for capillary filaments is best understood by examining how fluid wets and fills the microchannel.

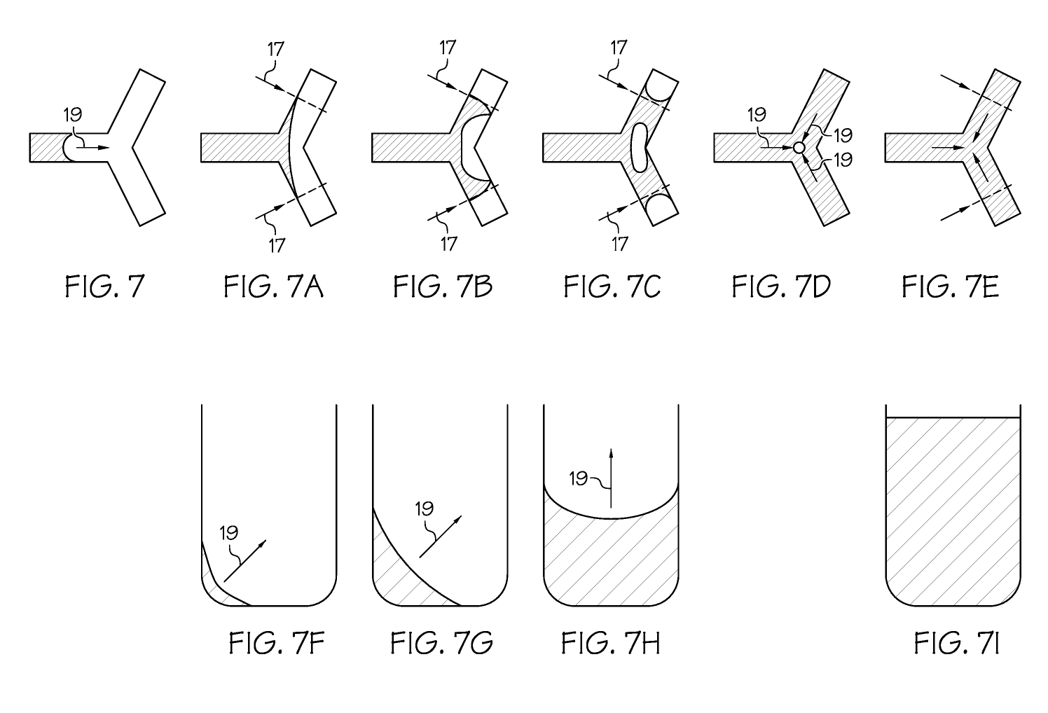

With reference to FIGS. 7 (1)-(6), the microchannel transports nanoliters of fluid through spontaneous capillary flow in the direction of the arrow(s) 19. The specific flow patterns described in FIG. 7 are an example of how flow can propagate in an open-microfluidic platform, and do not represent all possible propagation platforms possible with the present invention. In FIG. 7(1), the fluid wicks through a first channel and reaches a junction. Next, in FIG. 7(2) the fluid continues to flow along the corners of the junction due to capillary flow. In FIG. 7(3), the capillary forces cause the fluid to contact the opposite sidewalls, and a bubble remains in the middle of the junction, as shown in FIG. 7(4). As the fluid continues to move through the two channels, capillary forces cause the fluid to contact more of the surface of the opposite sidewalls as shown in FIG. 7(5). Finally, the bubble disappears as the fluid continues to move through the two channels, FIG. 7(6).

With reference to FIGS. 7A(2)-(4) & (6), which show a channel cross section from the direction of arrows 17, as fluid enters a u-channel, it propagates in a repeating pattern comprising four main steps: in FIG. 7A(2), a capillary filament occurs at the corners of the channels, and travels ahead of the bulk capillary flow, filling in the direction of the arrow 19; in FIG. 7A(3), the capillary filament has a concave meniscus and therefore due to Laplace pressure also fills the corner into the cross-section of the channel; in FIG. 7A(4) the capillary filament reaches the other channel side wall, and a new concave meniscus is formed which then further fills the channel due to Laplace pressure; and in FIG. 7A(6) the filled channel then supports bulk capillary flow, which follows additional capillary filaments traveling ahead of the bulk flow.

In the hex wicks disclosed herein, the capillary filaments propagate so quickly that they surround an entire hexagon perimeter before channel filling occurs. It should be noted that although the maximum volume of a hex wick with 10×15 μm channels that cover 10% of surface area ˜150 nL/cm², during use with a hydrogel or cellulose wicking pump it is unlikely the channels will be fully filled, and the volume during use is likely less than 100 nL/cm². By scaling the dimensions of the channels, the present invention can enable volumes of <10,000 nL/cm², <1,000 nL/cm², <100 nL/cm², or even <10 nL/cm². Using different configurations disclosed herein, the maximum wicking volume of the hex wick will also vary. For example, a hex wick with 10×10 μm channels covering 10% of the surface area would have a maximum wicking volume of less than 100 nL/cm². Similarly, if the hex wick includes more channels as a percentage of surface area, e.g., 20%, 25%, or 30%, the maximum wicking volume will be higher, e.g., up to 1000 nL/cm², up to 500 nL/cm², or up to 300 nL/cm². Hex wick compatibility with sensors may also influence configuration of the wick.

Because the hex wick requires the described capillary filaments to promote continuous wicking, choice of materials becomes a major challenge. A capillary filament can be understood by representing the corners of the channels as rounded v-grooves with dimensions discussed for previous examples, and can be modeled as:

$$sin α ( 1 + 2 α \frac{z}{w} ) < cos θ .$$

the same numbers described previously, and assuming a corner rounding radius of 1 μm, the necessary contact angle is <35°. Achieving this contact angle will require coating the microchannels with a functionalization coating to promote capillary filament propagation. Such a functionalization coating must meet certain criteria, namely, it first must be compatible with the analyte exchange blocking coating which covers the hex wick polymer. Second, the functionalization coating must be biologically compatible, and should be generally regarded as safe (GRAS) for skin contact during biofluid sensing applications, even if the functionalization coating becomes detached from the hex wick. Examples of thiols that would be suitable for such a purpose include monothiol thioglycolic acid (TGA), sodium 3-mercapto-1-propanesulfonate (MPS), both of which showed the required contact angle of <30°. Third, the functionalization coating should have long-term shelf stability. Materials showing better long-term stability include peptides, e.g., 5mer (2 cysteine groups, dithiol) and 7mer (3 cysteine groups, trithiol) peptides, with aspartic acid as the additional group to improve hydrophilicity. Using peptide functionalization coatings as disclosed allows the hex wick to have long term shelf stability of at least 30 days, and as long as 1 or 2 years. Stability may be enhanced by storage of the hex wick in nitrogen gas. Fourth, the functionalization coating should facilitate usage stability sufficient to cover most biofluid sensing applications. This means the functionalization coating should remain adhered to the analyte exchange blocking coating when exposed to the biofluid and/or a sensing surface (such as skin) for 8 hours, 24 hours, 7 days, or as long as 30 days.

In some embodiments, the functionalization coating includes a peptide-based SAM, which can very low contact angles (e.g., less than 30°) and is capable of retaining this contact angle for a period of days or more. To make a wicking collector with a peptide-based SAM, an epoxy-based photoresist is deposited onto a flexible substrate (e.g., a plastic such as PET). The photoresist may be negative (e.g., SU-8) or positive and should be capable of providing an accurate, high aspect ratio pattern. The flexible substrate is developed to have a hexagonal pattern through conventional microfabrication techniques. The hexagonal structure is then coated to reduce analyte exchange between analytes in the sample and the surface of the substrate. The blocking coating may be gold, silver, mercury, etc. The coated, patterned structure is soaked in a peptide solution to functionalize the surface with the thiols and form the hydrophobic peptide-based SAM on the surface.

Suitable peptides include, without limitation, peptides containing alternating residues with either charged moieties, e.g., aspartic acid (“D”), lysine (“K”), histidine (“H”), and/or glutamate (“E”), or thiols, e.g., cysteine acid (“C”). The amino acids may be native or non-native, L- or D-stereoisomers, beta-amino acids, etc. The amino acids may be functionalized to enable conjugation to other biomolecules using common linking groups (e.g., with amines, hydroxyls, carboxylic acids), and the peptides may have varying lengths. For example, the primary structure of a 7-mer peptide is DCDCDCD, and the primary structure of a 5-mer peptide is DCDCD, where D=aspartic acid and C=cysteine acid, both naturally occurring amino acids shown below. Peptides having different numbers of amino acids or other combinations of amino acids can be used to modify the surface and/or to create extremely stable linker groups. For example, a 9-mer peptide (DCDCDCDCD), may create a more stable and more hydrophilic SAM. Other embodiments may include peptides with the following compositions: ECECE, ECECECE, KCKCK, or KCKCKCK. In some other embodiments, the surface may be made by incorporation of hydrophobic amino acids (leucine, isoleucine, valine, alanine, phenylalanine, etc.).

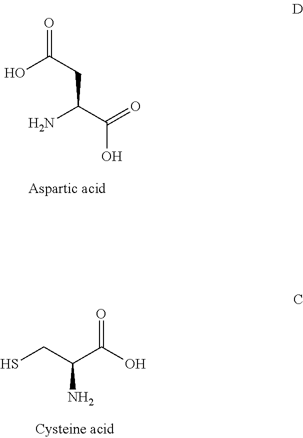 [SMILES: N[C@@H](CC(=O)O)C(=O)O ; N[C@@H](CS)C(=O)O]

The above-described configurations represent a basic foundation for either a simple device or a more complex device. Some embodiments of the disclosed invention may therefore include additional materials, components, designs, or other features for operation, as long as the device uses at least one wicking component, or operates at least in part by wicking pressure. More generally, regardless of how a wicking collector, a wicking pump, or a wicking coupler are configured, arranged, or omitted from a device of the present disclosure, the wicking pressure(s) are such that the sensor(s) is able to receive adequate biofluid to perform accurate measurements during device operation. In order to facilitate a more complete understanding of the embodiments of the invention, the following non-limiting examples are provided.

### Example 1

Under in vivo test conditions, the invention as disclosed achieved electrode response within 3 minutes after the initiation of sweat stimulation. This timing is the fastest sweat-to-sensor transport time currently known in the art, and roughly agrees with the modeled transport times. For example, a hex wick used as described has 10×15 μm channels at 10% of the area, and therefore ˜150 nL/cm² maximum volume. If the sweat generation rate is approximately 500 nL/min/cm² (as measured with a gravimetric sweat collector), then 1 cm² of the wick should fill up in 18 seconds (hex wick volume/sweat generation). The actual collection area used of 0.95 cm² should also provide an input sweat flow rate of 475 nL/min. Next, the maximum volume of the remainder of the hex wick is 60 nL, and the volume of the wicking coupler on the electrodes is ˜6% of total volume, ˜270 nL. The total volume is therefore 480 nL and the sensors should all respond within 500 nL/475 nL/minute, or approximately 60 seconds.

### Example 2

Peptides. 7-mer and 5-mer peptides containing alternating residues with either charged moieties (aspartic acid) or thiols (cysteine) were used to form peptide-based SAMs.

Patterned substrate preparation. A flexible PET substrate was first cleaned. The cleaning process included cooling the PET substrate to 10° C. in a Plasma-Preen® plasma cleaning/etching system. The sides of the PET substrate were taped to the plate in the Plasma-Preen® system. The PET substrate was treated with oxygen plasma for 2 min at 25% current. The treated substrate was left in a vacuum environment until the spin coating process.

To spin coat the treated substrate, the treated PET substrate was placed on a 3 mm thick acrylic carrier on a vacuum chuck. A layer of water between the PET substrate and acrylic carrier prevented the PET substrate from being spun off the carrier due to the van der Waals forces. An epoxy-based negative photoresist, Su-8, was heated for 20 min to 55° C. on a hot plate. No bubbles were in the photoresist liquid before it was poured onto the treated PET substrate. The substrate was spun at 500 rpm for 1:00 min at 500 acceleration and then at 3500 rpm for 35 s at 700 acceleration. Next, the coated substrate was subjected to a soft bake for 20 min at 70° C. on a hot plate and then allowed to cool to room temperature. The edge bead was removed. The coated substrate was then exposed. a) Expose 10 s using manual shutter; b) Rinse thoroughly with deionized water; c) Blow dry with N₂

Next, the substrate underwent a post-exposure bake (PEB). For 45 min, the substrate was baked on a hot plate at 70° C. The features were visible at the end of the PEB. The substrate was then allowed to cool to room temperature.

The substrate was then developed. The substrate was dipped in and out of propylene glycol methyl ether acetate (PGMEA) for 1 min followed by drying with N₂ gas. The substrate was rinsed again with fresh PGMEA. Next, the substrate was rinsed with water until the cloudiness disappeared and then rinsed again with isopropyl alcohol. Any cloudiness at this point may be indication of undissolved Su-8. The surface of the substrate was dried with N₂ gas. The developed substrate was subjected to a hard-bake overnight in an oven at 70° C. Gold was sputtered on the surface of the patterned substrate.

Peptide-based SAM preparation. Patterned substrates made using the preceding method were functionalized with thiols from three different materials—MPS, 7-mer, and 5-mer—which allowed for the formation of the SAMs. First, 70.9 g of 7-mer and 51.26 g of 5-mer were each mixed in 3 mL of a buffer solution (1×PBS) in an Eppendorf tube to make a 30 mMol solution. Also, a 30 mMol MPS mixture was made using 15 mg of sodium 3-mercapto-1-propanesulfonate powder with 3 mL of 1×PBS solution. The three solutions were mixed until they were clear to the eye. The gold-coated substrates were soaked in the different peptide solutions for 10 min so that the thiols deposited, forming a SAM. The SAM is hydrophilic, thus satisfying the condition for spontaneous capillary flow.

Results. Three of each kind of samples were all made at the same time and stored in different environments: (1) regular open atmosphere, (2) a nitrogen box, and (3) in a buffer solution with a potential of hydrogen (pH) similar to that of sweat.

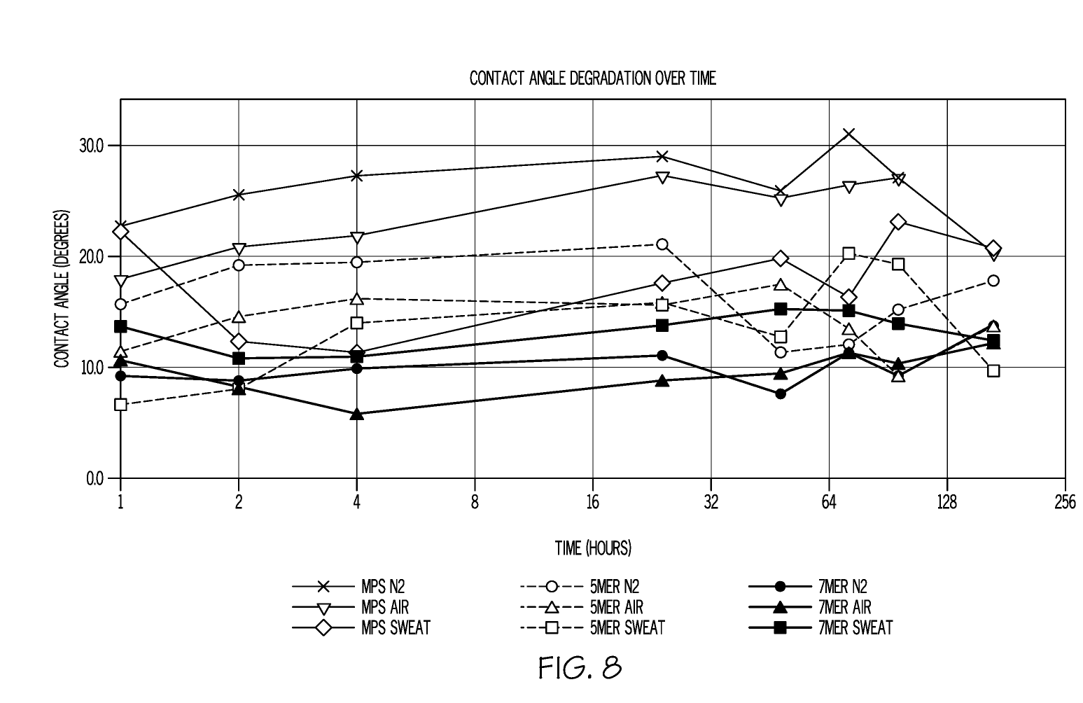

It was assumed that the 7-mer and 5-mer peptides had a beta-sheet secondary structure, which means that the thiols are all on the same side, and the charged residues are all on the same side opposite the side of the thiols. Accordingly, this arrangement allowed for a gold-sulfur interface between the SAM and the gold coating and a charged residue (carboxylate) water interface. The carboxylates made the surface hydrophilic (e.g., less than a 30° contact angle) at a pH of greater than 4.5. FIG. 8 shows the contact angle of the 7-mer and 5-mer stability in different environments and holding a super-hydrophilic contact angle for an entire week.

The various features discussed herein may be used alone or in any combination. Additional advantages and modifications will readily appear to those skilled in the art. The invention in its broader aspects is therefore not limited to the specific details, representative apparatus and methods and illustrative examples shown and described. Accordingly, departures may be made from such details without departing from the scope of the general inventive concept.

This has been a description of the present invention along with a preferred method of practicing the present invention, however the invention itself should only be defined by the appended claims.

What is claimed is:

1. A device, comprising:
a substrate having a surface that is hydrophilic and a plurality of open microchannels arranged in a networked pattern in the surface; and
a functionalization coating covering the plurality of open microchannels.

2. The device of claim 1, further comprising a blocking coating on the surface and the plurality of open microchannels.

3. The device of claim 1, wherein the microchannels comprise a volume of at least one of <10,000 nL/cm², <1,000 nL/cm², <100 nL/cm², <10 nL/cm².

4. The device of claim 1, wherein the networked pattern has a plurality of junctions among the microchannels, wherein each of the plurality of junctions includes at least three intersecting microchannels.

5. The device of claim 4, wherein the networked pattern is a hexagonal pattern.

6. The device of claim 1 wherein the functionalization coating is impermeable to water.

7. The device of claim 1, wherein the functionalization coating is comprised of one or more of the following: a monothiol thioglycolic acid; sodium 3-mercapto-1-propanesulfonate; a peptide, a 5mer peptide; and a 7mer peptide.

8. The device of claim 1, wherein the functionalization coating promotes a contact angle between a biofluid and a channel surface that is one of the following: less than 75 degrees; less than 66 degrees; less than 35 degrees; and less than 30 degrees.

9. The device of claim 1, further comprising one or more of the following in fluidic communication with at least a portion of the plurality of open microchannels: one or more wicking pumps, one or more sensors for measuring a characteristic of an analyte in a biofluid, and one or more wicking couplers.

10. The device of claim 9, further comprising: one or more of the following sensors: a volumetric sweat rate sensor, a micro-thermal flow rate sensor, a galvanic skin response sensor, a sweat conductivity sensor, an impedance sensor, and a capacitance sensor.

11. The device of claim 2, wherein the blocking coating comprises a hydrophilic gold layer.

12. The device of claim 1, wherein the device is configured to have a storage stability duration of one of the following: 30 days; 1 year; and 2 years.

13. The device of claim 1, wherein the device is configured to have a usage stability duration of one of the following: 1 day; 7 days; and 30 days.

14. The device of claim 1, wherein each of the plurality of open channels have a height-to-width aspect ratio of one of: >1:3, >1:2, >1:1, >1:1.5, >1:2, >1:3, >1.5:1, >2:1, or >3:1.

15. A method of forming a self-assembled monolayer (SAM) on a substrate, comprising:
modifying a plurality of peptides by attaching one or more of the following to each of the plurality of peptides: an amine molecule, or a thiol molecule;
attaching one or more of the following to a surface of the substrate: a plurality of graphene molecules, and a plurality of gold atoms; and
attaching the plurality of peptides to the surface of the substrate through one or more of the following: a plurality of amine to graphene bonds, and a plurality of thiol to gold bonds.

16. The method of claim 15, wherein each peptide includes an alternating sequence comprising a first amino acid residue and a second amino acid residue, wherein each first amino acid residue and each second amino acid residue contains a thiol molecule or an amine molecule.

17. The method of claim 15 wherein each peptide includes a sequence comprising a plurality of cysteine molecules, wherein the peptide includes a first side with an alpha helix, and wherein the cysteine molecules are arranged one side of the alpha helix.

18. The method of claim 15 wherein each peptide includes a sequence comprising a plurality of lysine molecules, wherein the peptide includes a first side with an alpha helix, and wherein the lysine molecules are arranged on one side of the alpha helix.

19. The method of claim 15, wherein each peptide is attached to a bio-recognition element.

20. The method of claim 19 where the bio-recognition element is bonded through a non-native amino acid coupling.

21. The method of claim 20 where the non-native coupling uses N-hydroxy-succinimide groups, malemide groups, alkyne groups, or azide groups.

22. The method of claim 15, further comprising treating the surface of the substrate with coating comprising a plurality of thiols before attaching the plurality of peptides to the surface.

23. The method of claim 22, wherein the coating further comprises one of the following: gold, silver, iron, mercury, or graphene.

24. The method of claim 16, wherein the first amino acid residue is an aspartic acid and the second amino acid residue is a cysteine acid.

25. The method of claim 15, wherein a primary structure of the peptide is one of the following, wherein “D” is an aspartic acid, “C” is a cysteine acid, “E” is a glutamate, and “K” is a lysine: DCDCD, DCDCDCD, ECECE, ECECECE, KCKCK, or KCKCKCK.

26. The method of claim 15, wherein the SAM maintains a fluid contact angle of less than 30° for a period of at least one day.

27. The method of claim 15, further comprising: patterning the substrate to form a plurality of channels.

28. The method of claim 27, wherein the plurality of channels form a pattern comprising a plurality of adjacent hexagons.

29. The method of claim 27, further comprising: transporting a fluid sample through the channels, wherein the SAM is hydrophobic.

30. A device, comprising:
a substrate including a surface; and
a self-assembled monolayer (SAM) attached to the surface, the SAM comprising: a plurality of peptides, wherein each peptide includes an alternating sequence comprising a first amino acid residue and a second amino acid residue, wherein each first amino acid residue and each second amino acid residue includes a charged moiety, and each first amino acid residue and each second amino acid residue is attached to a thiol.

31. The device of claim 30, further comprising a coating between the surface and the SAM.

32. The device of claim 31, where the coating comprises one of the following: gold, silver, or mercury.

33. The device of claim 30, wherein the first amino acid residue is aspartic acid and the second amino acid residue is cysteine acid.

34. The device of claim 30, wherein a primary structure of the peptide is one of the following, wherein “D” is an aspartic acid, “C” is a cysteine acid, “E” is a glutamate, and “K” is a lysine: DCDCD, DCDCDCD, ECECE, ECECECE, KCKCK, or KCKCKCK.

35. The device of claim 30, wherein the SAM maintains a fluid contact angle of less than 30° for a period of at least one day.

36. The device of claim 30, wherein the substrate comprises a plurality of channels.

37. The device of claim 36, wherein the plurality of channels form a honeycomb shape.

38. The device of claim 36, wherein the SAM is hydrophobic, and the plurality of channels is configured to transport a biofluid.
